(19) United States
(12) Patent Application Publication
(10) Pub. No.: US 2024/0299330 A1
(43) Pub. Date: Sep. 12, 2024

(54) MCT FORMULATIONS FOR IMPROVING COGNITIVE FUNCTIONS AND METHODS OF MAKING AND USING SUCH FORMULATIONS

(71) Applicant: SOCIETE DES PRODUITS NESTLE S.A., Vevey (CH)

(72) Inventor: NANCY STEVENSON, Linwood, NJ (US)

(21) Appl. No.: 18/005,990

(22) PCT Filed: Jul. 20, 2021
(86) PCT No.: PCT/EP2021/070290

§ 371 Date: Jan. 19, 2023

Related U.S. Application Data
(60) Provisional application No. 63/055,552, filed on Jul. 23, 2020.

(51) Int. Cl. A61K 31/23; A23D 9/007; A61K 47/36; A61K 47/38

(52) U.S. Cl. CPC A61K 31/23; A23D 9/007; A61K 47/36; A61K 47/38

(57) ABSTRACT

The present disclosure provides compositions containing medium chain triglycerides (MCTs) and also containing a food matrix into which at least a portion of the MCTs are formulated. The compositions include specific gums which provide stability and good mouthfeel. The compositions can improve cognitive functioning, support memory and/or recall, provide energy and/or ketones to the brain, and/or prevent and/or treat mild cognitive impairment (MCI).

### CROSS REFERENCE TO RELATED APPLICATIONS

The present application is a National Stage of International Application No. PCT/EP2021/070290, filed on Jul. 20, 2021, which claims priority to U.S. Provisional Patent Application No. 63/055,552, filed on Jul. 23, 2020, the entire contents of which are being incorporated herein by reference.

### BACKGROUND

The present disclosure generally relates to compositions comprising medium chain triglycerides (MCTs) and further comprising a food matrix into which at least a portion of the MCTs are formulated. The compositions include specific gums which provide stability and good mouthfeel. The compositions can improve cognitive functioning, support memory and/or recall, provide energy and/or ketones to the brain, and/or prevent and/or treat mild cognitive impairment (MCI).

The two main ketones, beta-hydroxybutyrate (BHB) and acteto actetate (AcA), represent an important alternative source of energy for extrahepatic tissues like brain, heart or skeletal muscle. Moreover, accumulating evidence suggests that ketones might also have a signaling role, either direct or indirect. Products aimed at increasing blood ketones have potential therapeutic benefits in several conditions including, but not limited to, epilepsy, neurological and neurodegenerative diseases, heart failure, inborn errors of metabolism, obesity, type 2 diabetes, cancer, exercise performance, and nonalcoholic fatty liver disease (NAFLD) such as nonalcoholic steatohepatitis (NASH).

BHB and AcA are actively transported to the brain by the monocarboxylic transporter 1 (MCT1), resulting in brain levels directly proportional to their blood concentrations. Therefore, products that provide a more sustained plasma level of ketones are anticipated to have a longer effect (longer plasma ketones half-life Tki/2) compared to products that raise blood ketones for a shorter time span (shorter half-life).

Medium-chain triglycerides (MCTs) are efficient ketone precursors when administered by oral bolus. They are rapidly digested, and the resultant free medium chain fatty acids (MCFAs) are absorbed efficiently by the portal vein to reach the liver where they are extensively metabolized to ketones, bypassing the normal long-chain fatty acid digestion and absorption processes. Their specific formulation can affect ketogenesis efficiency and gastrointestinal tolerability.

Brain energy rescue is a potential strategy to reduce cognitive decline in mild cognitive impairment (MCI) and Alzheimer's disease (AD). Impairment of some particular cognitive functions, such as episodic memory and language skills, are predictive of MCI and AD. In those at increased risk of early- or late-onset AD, brain glucose uptake is already lower before the onset of the mild cognitive deficit associated with MCI. Hence, a pre-symptomatic brain energy (glucose) deficit of about 10% is present which is sufficient to be contributing to cognitive decline in MCI.

MCTs have been included for various purposes in food compositions for animals or humans. MCTs have been added to “wet” foods such as canned food and other prepared food and have been used to some extent in, or as coatings on, dry food. Practical considerations appear to limit the amount of MCTs that can be used, particularly for dry foods. For example, while wet food compositions may incorporate a significant amount of MCTs, they may be subject to separation, flavor issues, and other problems. For dry foods, such as extruded and baked products, the compositions are strictly subject to limits wherein the MCTs cannot be increased above a certain threshold in many products. MCTs have thus been used as a coating or surface application for such foods. Surface application, such as coating, poses practical problems for packaging and reduces consumer appeal. For example, consumption or administration of such food compositions is far more cumbersome if a coating must be poured on or mixed in at the time of consumption or if the dry food has an oily appearance or feel. In addition, large quantities of MCTs not integrally incorporated into the food matrix can cause problems with palatability and tolerance of such food compositions.

Chemically, MCTs comprise a group of triglycerides having three medium chain length (about 6-12 carbon) fatty acid molecules esterified to a glycerol molecule. Such compositions are different, both physically and chemically, from the majority of fats typically used in food technology for formulating food products. MCTs tend to be shorter and have different functional properties for processing than their longer-chain counterparts. For example, MCTs are typically liquid at room temperature, unlike many other functional fats used by food technologists.

In addition to having different properties that must be considered for formulation of food compositions, MCTs pose other challenges for formulators. When attempting to formulate a food with a specified macronutrient content, addition of MCTs may require removal of other fat sources. Many food compositions are formulated such that the fat provides significant satiety, flavor, mouthfeel, texture, and other desirable functional qualities that the consumer is seeking from a particular food product. Thus, mere substitution of one fat for another is not a practical solution because the formulation of a food product involves complex interactions between the various ingredients or components. In fact, MCT-based formulas present on the market can have sedimentation as well as poor mouthfeel, which are not desirable by consumers.

### SUMMARY

The present inventors surprisingly and unexpectedly discovered that gums, especially some specific gum(s), can provide MCT based formulas stability by reducing sedimentation and also providing good mouthfeel.

Accordingly, in a non-limiting embodiment, the present disclosure provides a composition comprising: medium chain triglycerides (MCTs); and at least one gum selected from the group consisting of xanthan gum, gellan gum, carrageenan (iota), carboxymethyl cellulose (CMC), and microcrystalline cellulose (MCC). The at least one gum may comprise gellan gum and carrageenan iota; or at least one of carboxymethyl cellulose (CMC) or microcrystalline cellulose (MCC).

The MCTs may comprise from 51 wt % to 90 wt % of octanoic acid. The MCTs may comprise from 51 wt % to 70 wt % of octanoic acid. The MCTs may comprise from 71 wt % to 90 wt % of octanoic acid. The MCTs may comprise 60 wt % of octanoic acid. The MCTs may further comprise decanoic acid. In one embodiment, a weight ratio of the octanoic acid and the decanoic acid is about 60:40.

The composition can further comprises at least 1 wt % milk protein concentrate (MPC), for example, about 5 wt % or about 7.5 wt % of MPC. The composition can further comprises at least 4 g protein/100 g of the composition. The composition can further comprise proteins in a weight ratio of at least 0.1 g protein/1.0 g of the MCTs. The composition further comprises proteins in a weight ratio of at least 0.4 g protein/1.0 g of the MCTs.

The composition can optionally further comprise (i) carbohydrates in a weight ratio of at least 0.1 g carbohydrate/1.0 g of the MCTs, and/or (ii) lipids, other than the MCTs, in a weight ratio of at least 0.1 g lipids/1.0 g of the MCTs.

The composition can have a pH from about 6.6 to about 7.5.

The composition can be in a form selected from the group consisting of a beverage, mayonnaise, salad dressing, margarine, low-fat spread, dairy product, cheese spread, processed cheese, dairy dessert, flavoured milk, cream, fermented milk product, cheese, butter, condensed milk product, ice cream mix, soya product, pasteurised liquid egg, bakery product, confectionary product, confectionary bar, chocolate bar, high fat bar, liquid emulsion, spray-dried powder, freeze-dried powder, UHT pudding, pasteurised pudding, gel, jelly, yoghurt, a food with a fat-based or water-containing filling, and combinations thereof.

The composition may be an oral nutritional composition, a nutritional supplement, an oral nutritional supplement, a medical food, a food supplement, a food product, or a food for special medical purpose (FSMP).

The composition may be in a form of a solid powder, a powdered stick, a capsule, or a solution.

In another non-limiting embodiment, the present disclosure also provides a method of improving sedimentation and/or mouthfeel attributes of a composition comprising medium chain triglycerides (MCTs), the method comprising providing to the composition at least one gum selected from the group consisting of xanthan gum, gellan gum, carrageenan (iota), carboxymethyl cellulose (CMC), and microcrystalline cellulose (MCC).

In other embodiments, the composition is used in a method of supporting memory and/or recall in an individual; a method of providing energy and/or ketones to the brain of an individual; or a method of preventing and/or treating mild cognitive impairment (MCI) in an individual.

The individual may have mild cognitive impairment (MCI). The individual suffers from at least one of deficit in memory, impaired thinking skill comprising inability to make sound decisions and poor judgment, depression, or anxiety. The individual can have or suffer from a brain energy deficiency condition or disease, neurological condition, and/or cognitive impairment.

In some embodiments, the compositions can be administered to an individual having a condition selected from the group consisting of epilepsy, a neurological disease, a neurodegenerative disease, heart failure, inborn errors of metabolism, obesity, types 2 diabetes, nonalcoholic fatty liver disease (NAFLD), nonalcoholic steatohepatitis (NASH), cancer, a brain energy deficiency condition, a migraine, a memory disorder, an age-related memory disorder, a brain injury, a stroke, amyloid lateral sclerosis, multiple sclerosis, cognitive impairment, mild cognitive impairment (MCI), cognitive impairment post-intensive care, age-induced cognition impairment, Alzheimer's disease, Parkinson's disease, Huntington's disease, an inherited metabolic disorder, bipolar disorder, schizophrenia, and combinations thereof.

Additional features and advantages are described in, and will be apparent from, the following Detailed Description.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of and “consisting of the disclosed components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal benefitting from ketones. While the term “individual” is often used herein to refer to a human, the present disclosure is not so limited. Accordingly, the term “individual” refers to any animal, mammal or human that can benefit from the methods and compositions disclosed herein.

The relative terms “improved,” “increased,” “enhanced” and the like refer to the properties or effects of the composition containing MCTs in a food matrix (disclosed herein) relative to a composition with an identical formulation except for a lower amount of protein and/or carbohydrate. The terms “maintained” and “sustained” mean that a characteristic of an individual, such as neurologic health, cognitive function or exercise performance, is approximately the same as the average level for the preceding week, the average level for the preceding month, or the average level for the preceding year.

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “prevent” and “prevention” mean to administer a composition as disclosed herein to a subject is not showing any symptoms of the condition to reduce or prevent development of at least one symptom associated with the condition.

As used herein, “cognitive function” refers to any mental process that involves symbolic operations, e.g., perception, memory (free recall), executive function, processing speed, attention, speech comprehension, speech generation, language, reading comprehension, creation of imagery, learning, and reasoning, preferably at least memory.

The terms “food,” “food product” and “food composition” mean a composition that is intended for ingestion by an individual, such as a human, and that provides at least one nutrient to the individual. The term “food matrix” means the physical structure of the food composition, which can be liquid, solid, or semi-solid in various embodiments. “Food” and its related terms include any food, feed, snack, food supplement, treat, meal substitute, or meal replacement, whether intended for a human or an animal. Animal food includes food or feed intended for any domesticated or wild species. In preferred embodiments, a food for an animal represents a nutritionally complete food or dietary composition, e.g., a pelleted, extruded, or dry food. Examples of such animal foods include extruded pet foods such as foods for dogs and cats.

The term “food for special medical purpose (FSMP)” refers to formula foods specially processed and prepared in order to meet special needs for nutrient or diet of those suffering from food intake restriction, disorder of digestive absorption, disorder of metabolic or certain diseases. Such foods shall be used alone or together with other foods under the guidance of a doctor or clinical nutritionist. FSMP is special dietary food, not medicine, but not ordinarily eaten by normal people. It is specially developed by clinicians and nutritionists based on scientific facts after extensive medical research.

The term “oral nutritional supplement (ONS)” refers to sterile liquids, semi-solids or powders, which provide macro and micro nutrients. They are widely used within the acute and community health settings for individuals who are unable to meet their nutritional requirements through oral diet alone.

A triglyceride (also known as a triacylglycerol or a triacylglyceride) is an ester that is derived from glycerol and three fatty acids. Fatty acids may be either unsaturated or saturated. Fatty acids which are not attached to other molecules are referred to as free fatty acids (FFA).

A medium-chain triglyceride (MCT) is a triglyceride in which all three fatty acid moieties are medium-chain fatty acid moieties. As defined herein, medium-chain fatty acids (MCFA) are fatty acids that have 6 to 14 carbon atoms, preferably 6 to 12 carbon atoms. Medium-chain fatty acids with 8 carbon atoms may be referred to herein as “C8 fatty acids” or “C8.” Medium-chain fatty acids with 10 carbon atoms may be referred to herein as “C10 fatty acids” or “C10.”

The term “fatty acid moiety” refers to the part of the MCT that originates from a fatty acid in an esterification reaction with glycerol. In a non-limiting example, an esterification reaction between glycerol and only octanoic acid would result in a MCT with octanoic acid moieties. In another non-limiting example, an esterification reaction between glycerol and only decanoic acid would result in a MCT with decanoic acid moieties.

Octanoic acid (also known as caprylic acid) is a saturated fatty acid of the formula CH3(CH2)6COOH.

Decanoic acid (also known as capric acid) is a saturated fatty acid of the formula CH3(CH2)8COOH.

### EMBODIMENTS

An aspect of the present disclosure is a composition comprising medium-chain triglycerides (MCTs) with improved sedimentation and mouthfeel attributes. The composition may be an oral nutritional composition, a nutritional supplement, an oral nutritional supplement, a medical food, a food supplement, a food product, or a food for special medical purpose (FSMP). The composition may be in a form of a solid powder, a powdered stick, a capsule, or a solution.

The composition preferably comprises a food matrix into which at least a portion of the MCTs is formulated, and a particularly preferred non-limiting embodiment of the composition is a liquid such as a beverage. The composition can be also in the form of a powder that can be readily dissolved into a liquid, such as water, prior to ingestion.

The MCTs comprise three fatty acid moieties, each of which independently has between 6-12, 6-11, 6-10, 7-12, 7-11, 7-10, 8-12, 8-11 or 8-10 carbon atoms. In an embodiment, at least a portion of the MCTs contain one or more octanoic acid moieties. In an embodiment, at least a portion of the MCTs contain one or more decanoic acid moieties. In an embodiment, at least a portion of the MCTs contain one or more octanoic acid moieties and one or more decanoic acid moieties.

In one embodiment, the MCTs comprises from 51 wt % to 90 wt % of octanoic acid. In one embodiment, the MCTs comprises from 51 wt % to 70 wt % of octanoic acid. In one embodiment, the MCTs comprises from 71 wt % to 90 wt % of octanoic acid. In one embodiment, the MCTs comprises 60 wt % of octanoic acid. In one embodiment, a weight ratio of the octanoic acid and the decanoic acid is 60:40.

The composition may further comprise at least one MCT maintaining component, which can be, for example, starch, fiber, gluten meal, protein, an emulsifier, a stabilizer, a gum and/or a gelling agent. The MCT maintaining component may comprise one or more of a component that can alter the structure of water in the composition, a component that can bind fat in the food composition, and/or a component that can emulsify or stabilize the fat in the food composition. Such components may include components that can bind water in a food matrix, bind fat, emulsify fat, stabilize emulsions of water and fat, or similarly maintain the MCT in association with the food matrix. Including one or more of such components can, for example, increase the actual retention of MCT in the food matrix and, in some embodiments, provide improvements in other functional properties such as maintaining desirable density, volume, texture, hardness, crunchiness, or the like in foods such as extruded food compositions.

Such components may be from any source whether natural or synthetic. Natural source includes all such sources whether animal, vegetable, or microbial in nature. Starches, or starch-like molecules, as well as gums, can also be from a variety of photosynthetic sources in the plant or microbial kingdom.

For example, the composition may optionally further comprise a gum and/or an emulsifier. Including the gum(s) and/or emulsifier(s) can provide the composition with improved sedimentation and mouthfeel attributes. The gum can comprise xanthan gum, gellan gum, carrageenan iota, and/or cellulose gel/gum. Preferably, the composition comprises a blend of gellan gum and iota carrageenan; and/or cellulose gel/gum, for example, carboxymethyl cellulose (CMC) and/or microcrystalline cellulose (MCC). In an embodiment, the composition can comprise a blend of about 85 wt % MCC and about 15 wt % CMC, such as in some Avicel® products. The emulsifier can comprise, for example, one or more of soy lecithin, sunflower lecithin, or mono- and di-glycerides.

The gum(s) and/or emulsifier(s) can stabilize the MCT oil by reducing or eliminating sedimentation and excessive creaming of the composition in the liquid form, or if in a dry form, when dissolved in a fluid (e.g., water). For example, the composition may include a protein, which can emulsify at least a portion of the MCT oil, and the added gum can help keep the protein suspended and thus prevent sedimentation. The gum(s) can create a smooth mouthfeel, avoiding chalkiness in mouth that can be perceived negatively in terms of sensory. Further in this regard, the protein can function as an emulsifier for the MCT oil such that any additional emulsifier can be optionally omitted, and the gum can stabilize the protein and prevent protein sedimentation.

In an embodiment, the composition is administered to an individual in a serving that provides at least about 5 g MCTs, for example at least about 10 g MCTs, for example about 15 g MCTs. In an embodiment, the composition is administered to an individual in a daily dosage comprising from about 15 g to about 45 g MCTs; preferably in a daily dosage comprising about 30 g MCTs. In an embodiment, at least two servings of the composition are administered daily, each serving comprising about 15 g MCTs; preferably two servings of the composition are administered daily, although some embodiments can include more than two servings of the composition.

The composition may further comprise protein. The protein can be milk protein concentrate (MPC). The composition may comprise at least about 1 wt % MPC, for example, at least about 2 wt % MPC, from about 2 wt % to about 20 wt %, from about 2% to about 15 wt %, from about 2 wt % to about 10 wt %, from about 2 wt % to about 6 wt %, from about 2 wt % to about 5 wt %, from about 5 wt % to about 8 wt %, from about 5 wt % to about 7.5 wt %, about 5 wt %, or about 7.5 wt %.

The protein can be in weight ratio relative to the MCTs of at least about 0.1 g protein/1.0 g MCTs, preferably at least about 0.4 g protein/1.0 g MCTs, more preferably at least about 0.8 g protein/1.0 g MCTs, more preferably at least about 1.0 g protein/1.0 g MCTs, even more preferably at least about 1.5 g protein/1.0 g MCTs, most preferably at least about 1.7 g protein/1.0 g MCTs.

Optionally, the composition may further comprise a stabilizer, which can be stabilizing minerals, such as potassium citrate, for example, in embodiments of the composition that comprise a high protein content, for example, higher than 7.5% MPC. Non-limiting examples of suitable stabilizers include one or more citrates and/or one or more phosphates, which can bind with free magnesium and free calcium to stabilize the protein. Additionally or alternatively, a pre-heat treatment can denature the protein (e.g., whey) before UHT. Optionally the composition may further comprise carbohydrate and/or other lipids in addition to the MCTs.

If carbohydrate is present, the carbohydrate is preferably in weight ratio relative to the MCTs of at least about 0.3 g carbohydrate/1.0 g MCTs, preferably at least about 1.0 g carbohydrate/1.0 g MCTs, more preferably at least about 2.0 g carbohydrate/1.0 g MCTs, even more preferably at least about 3.0 g carbohydrate/1.0 g MCTs, yet more preferably at least about 4.0 g carbohydrate/1.0 g MCTs, most preferably at least about 4.7 g carbohydrate/1.0 g MCTs.

If lipid other than the MCTs is present, the lipid other than MCTs is preferably in a weight ratio relative to the MCTs of at least about 0.1 g lipid/1.0 g MCTs, at least about 0.2 g lipid/1.0 g MCTs, preferably at least about 0.3 g lipid/1.0 g MCT, at least about 0.4 g lipid/1.0 g MCT, at least about 0.6 g lipid/1.0 g MCT, at least about 0.8 g lipid/1.0 g MCT or at least 1.0 g lipid/1.0 g MCT. In an embodiment, if lipid other than MCTs is present the lipid other than MCT may be present at a ratio lipid other than MCT:MCTs of from 0.1:2.0 to 2.0:1.0, preferably 0.1:1.0 to 1.0:2.0.

The MCTs is preferably 1-50 wt. % of the composition, for example 1-30 wt. %, 1-10 wt. %, 2-10 wt. %, 3-10 wt. %, 4-10 wt. %, 5-10 wt. %, 6-10 wt. %, 7-10 wt. % or 8-10 wt. % of the composition. In an embodiment in which the composition is a liquid, the composition can comprise at least about 40 g MCTs/L, preferably at least about 50 g MCTs/L, more preferably at least about 75 g MCTs/L, even more preferably at least about 100 g MCTs/L, most preferably at least about 120 g MCTs/L. The MCTs can be in the liquid at a level up to about 250 g/L, preferably up to about 200 g MCTs/L, more preferably up to about 175 g MCTs/L, most preferably up to about 150 g MCTs/L.

In an embodiment, the composition can comprise at least about 4.0 g protein/100 g of the composition, for example, about 4.2 g protein/100 g, about 6.0 g protein/100 g, at least about 6.0 g protein/100 g, about 6.3 g protein/100 g, about 6.5 g protein/100 g, at least about 6.5 g protein/100 g, at least about 6.8 g protein/100 g of the composition.

In an embodiment in which the composition is a liquid, the composition can comprise at least about 40 g protein/L, for example, about 42 g protein/L, about 60 g protein/L, at least about 60 g protein/L, about 63 g protein/L, about 65 g protein/L, at least about 65 g protein/L, at least about 68 g protein/L.

In an embodiment in which the composition is a liquid, the composition can comprise at least about 36 g carbohydrate/L, preferably at least about 50 g carbohydrate/L, more preferably at least about 75 g carbohydrate/L, even more preferably at least about 100 g carbohydrate/L, yet more preferably at least about 150 g carbohydrate/L, most preferably at least about 188 g carbohydrate/L.

Preferably the composition contains one or more natural sources that provide at least a portion of the MCTs. Non-limiting examples of suitable natural sources of MCTs include coconuts, coconut oil, palm kernels, and palm kernel oils. For example, decanoic acid and octanoic acid form about 5-8% and 4-10% of the fatty acid composition of coconut oil, respectively.

Additionally or alternatively, at least a portion of the MCTs may be synthesized by esterification of glycerol with one or more medium-chain fatty acids (MCFA) with a tail of 6 to 12 carbon atoms. For example, a homotriglyceride comprising three fatty acid moieties each with 8 carbon atoms can be synthesized by esterification of glycerol with C8 fatty acids (e.g., octanoic acid), and a homotriglyceride comprising three fatty acid moieties each with 10 carbon atoms can be synthesized by esterification of glycerol with C10 fatty acids (e.g., decanoic acid).

In an embodiment, the composition comprises MCTs comprising at least one octanoic acid moiety or decanoic acid moiety, and the composition is free from or substantially free from any other triglycerides. As used herein, the term “free from any other triglycerides” means that the composition does not comprise any triglycerides that do not contain at least one octanoic acid moiety or decanoic acid moiety. As used herein, the term “substantially free from any other triglycerides” means that the composition may contain traces of other triglycerides, i.e., less than 5 mol %, preferably less than 3 mol %, more preferably less than 2 mol %, even more preferably less than 1 mol % or most preferably less than 0.5 mol %.

After oral absorption, MCTs are metabolized to free fatty acids and further metabolized to ketones. The free fatty acids are initially metabolized to 13-hydroxybutyrate (BHB) and then aceto acetate (AcA). MCFA and ketones can be produced in various amounts in bodily fluids depending on the MCT utilized, and they may be used as an alternative source of energy to glucose or to supplement the energy derived from glucose.

Ketones can be transported to the brain by, for example, monocarboxylic transporter 1 (MCT1) where they are mainly metabolized by neurons. Free fatty acids, such as C8 free fatty acids and C10 free fatty acids, can reach the brain by diffusion where they are mainly metabolized by astrocytes.

Oral administration of the composition to the subject provides one or more of ketones, C8 fatty acids, or C10 fatty acids to a bodily fluid of that subject. The exposure of the subject to ketones and/or specific fatty acids (e.g., C8 or C10 fatty acids) can be quantified by measuring the levels of ketones and/or specific fatty acids in the subject's plasma, e.g., over 8 hours following oral administration. The exposure of a subject to a ketone and/or specific fatty acids may be calculated by determining the area under the curve (AUC) in a plot of concentration of ketone and/or fatty acid in a bodily fluid e.g., blood plasma, against time (e.g., over 8 or 24 hours). Biological fluids can be treated prior to analysis with organic solvent to precipitate protein and reconstituted in a mass spectrometry (MS) compatible solvent. Levels of ketone bodies and medium chain fatty acids can be assessed using liquid chromatography coupled to high resolution mass spectrometry (LC-MS). In particular, f3-hydroxybutyrate (BHB), aceto acetate (AcA), and specific fatty acid concentrations can be quantitatively measured using an external calibration methodology.

In an embodiment, the protein is selected from the group consisting of dairy based proteins, plant based proteins, animal based proteins, artificial proteins, or combinations thereof

Dairy based proteins include, for example, casein, casein hydrolysates, caseinates (e.g., all forms including sodium, calcium, potassium caseinates), whey hydrolysates, whey (e.g., all forms including concentrate, isolate, demineralized), milk protein concentrate, and milk protein isolate. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, and pulses. Animal based proteins may include, for example, beef, poultry, fish, lamb, seafood, pork, egg, or combinations thereof.

In an embodiment the protein source is a dairy based protein. In an embodiment the dairy based proteins are selected from the group consisting of casein, caseinates, casein hydrolysates, whey, whey hydrolysates, milk protein concentrate, milk protein isolate, or combinations thereof

The composition may further comprise one or more additional components such as minerals; vitamins; salts; or functional additives including, for example, palatants, colorants, emulsifiers, antimicrobial or other preservatives. Non-limiting examples of suitable minerals for the compositions disclosed herein include calcium, phosphorous, potassium, sodium, iron, chloride, boron, copper, zinc, magnesium, manganese, iodine, selenium, chromium, molybdenum, fluoride and any combination thereof. Non-limiting examples of suitable vitamins for the compositions disclosed herein include water-soluble vitamins (such as thiamin (vitamin B1), riboflavin (vitamin B2), niacin (vitamin B3), pantothenic acid (vitamin B5), pyridoxine (vitamin B6), biotin (vitamin B7), myo-inositol (vitamin B8), folic acid (vitamin B9), cobalamin (vitamin B12), and vitamin C) and fat-soluble vitamins (such as vitamin A, vitamin D, vitamin E, and vitamin K) including salts, esters or derivatives thereof. Inulin, taurine, carnitine, amino acids, enzymes, coenzymes, and any combination thereof may be included in various embodiments.

In one embodiment, the composition further comprises at least one of pyridoxine (vitamin B6), folic acid (vitamin B9), or cobalamin (vitamin B12). Folate (vitamin B9) supplementation is beneficial for memory, better cognitive function, and psychomotor speed. Vitamin Bi2 deficiency is primarily related to altered absorption in the elderly. Vitamin B12 and folic acid deficiencies result in symptoms such as low mood, fatigue, irritability. Further, folate and vitamin B12 are required for the synthesis of neurotransmitters.

The composition may further comprise one or more agents that promote or sustain general neurologic health or further enhance cognitive function. Examples of such agents include choline, phosphatidylserine, alpha-lipoic acid, CoQ10, acetyl-L-carnitine, omega-3 fatty acid, herbal extracts (such as Gingko biloba, Bacopa monniera, Convolvulus pluricaulis and Leucojum aestivum).

In some embodiments, the composition has a pH from about 6.6 to about 7.5, preferably, from about 6.7 to about 7.0, from about 6.75 to about 7.0, more preferably from about 6.8 to about 7.0, from example, about 6.8, about 6.85, about 6.9, about 6.95, about 6.99, about 7.0.

The pH of the composition can be adjusted by adding an acid or an alkali. For example, citric acid can be used to decrease the pH, and sodium hydroxide and/or potassium hydroxide can be used to increase the pH of the composition.

The composition may be in the form of a medical food. The term “medical food” as used herein refers to a food product specifically formulated for the dietary management of a medical disease or condition; for example, the medical disease or condition may have distinctive nutritional needs that cannot be met by normal diet alone. The medical food may be administered under medical supervision. The medical food may administered orally or as a tube feed. The term “tube feed” refers to a product which is intended for introducing nutrients directly into the gastrointestinal tract of a subject by a feeding tube. A tube feed may be administered by, for example, a feeding tube placed through the nose of a subject (such as nasogastric, nasoduodenal, and nasojejunal tubes) or a feeding tube placed directly into the abdomen of a subject (such as gastrostomy, gastroj ej unostomy, or j ej unostomy feeding tube).

The composition may be in the form of a nutritional composition or a nutritional supplement. The term “nutritional supplement” refers to a product which is intended to supplement the general diet of a subject.

The composition may be in the form of a complete nutritional product. The term “complete nutritional product” refers to a product which is capable of being the sole source of nutrition for the subject.

In various embodiments, the composition may be in the form of a beverage, mayonnaise, salad dressing, margarine, low fat spread, dairy product, cheese spread, processed cheese, dairy dessert, flavored milk, cream, fermented milk product, cheese, butter, condensed milk product, ice cream mix, soya product, pasteurized liquid egg, bakery product, confectionary product, confectionary bar, chocolate bar, high fat bar, liquid emulsion, spray-dried powder, freeze-dried powder, UHT pudding, pasteurized pudding, gel, jelly, yoghurt, or a food with a fat-based or water-containing filling.

In an embodiment, the composition may be an infant formula. In yet other embodiments, the composition may be used to coat a food, snack, pet food, or pet treat.

The compositions disclosed herein may be administered to a subject enterally or parenterally. Preferably, the composition is administered enterally. For example, the composition may be administered in the form of a food stuff or a supplement. Enteral administration may be oral, gastric, and/or rectal. Preferably the composition is administered orally.

The subject may be a mammal such as a human, canine, feline, equine, caprine, bovine, ovine, porcine, cervine or a primate. Preferably the subject is a human. In an embodiment, the subject is an infant. The infant may, for example, be a human such as a newborn infant (i.e., a baby under 28 days of age) or a premature infant (i.e., a baby born before 37 completed weeks of gestation).

In an embodiment, the subject is an aging subject. For instance, a subject may be an aging subject when it has reached 40, 50, 60, 66, 70, 75, or 80% of its likely lifespan. A determination of lifespan may be based on actuarial tables, calculations, or estimates, and may consider past, present, and future influences or factors that are known to positively or negatively affect lifespan. Consideration of species, gender, size, genetic factors, environmental factors and stressors, present and past health status, past and present nutritional status, and stressors may be taken into consideration when determining lifespan. The aging subject may, for example, be a human subject over the age of 40, 45, 50, 55, 60, 65, 70, 75, 80, 85, 90, 95 or 100 years old.

In an embodiment, the subject is a subject diagnosed with mild cognitive impairment or suffering from memory complains. The subject may need improving cognition such as particularly episodic memory, executive function, and language. The subject may have or suffer from a brain energy deficiency condition or disease, neurological condition, and/or cognitive impairment.

All references herein to treatment include curative, palliative and prophylactic treatment. Treatment may also include arresting progression in the severity of a disease. Both human and veterinary treatments are within the scope of the present disclosure.

Free fatty acids and ketones produced from MCTs can provide an alternative energy source to glucose to supplement or replace the energy in cells such as astrocytes, myocytes, cardiomyocytes, or neuronal cells.

Brain tissue consumes a large amount of energy in proportion to its volume. In an average healthy subject, the brain obtains most of its energy from oxygen-dependent metabolism of glucose. Typically, the majority of the brain's energy is used to help neurons or nerve cells send signals and the remaining energy is used for cell-health maintenance. A deficiency in brain energy, for example caused by impairment of glucose utilization, can result in neuronal hyperactivity, seizures and cognitive impairments.

Examples of brain energy deficiency conditions or diseases include: migraine, memory disorder, age-related memory disorder, brain injury, neurorehabilitation, stroke and post-stroke, amyloid lateral sclerosis, multiple sclerosis, cognitive impairment, mild cognitive impairment (MCI), cognitive impairment post-intensive care, age-induced cognition impairment, Alzheimer's disease, Parkinson's disease, Huntingdon's disease, inherited metabolic disorders (such as glucose transporter type 1 deficiency syndrome and pyruvate dehydrogenase complex deficiency), bipolar disorder, schizophrenia, and/or epilepsy.

As used herein, the term “neurological condition” refers to a disorder of the nervous system. Neurological conditions may result from damage to the brain, spinal column or nerves, caused by illness or injury. Non-limiting examples of the symptoms of a neurological condition include paralysis, muscle weakness, poor coordination, loss of sensation, seizures, confusion, pain and altered levels of consciousness. An assessment of the response to touch, pressure, vibration, limb position, heat, cold, and pain as well as reflexes can be performed to determine whether the nervous system is impaired in a subject.

Some neurological conditions are life-long, and the onset can be experienced at any time. Other neurological conditions, such as cerebral palsy, are present from birth. Some neurological conditions, such as Duchenne muscular dystrophy, commonly appear in early childhood, while other neurological conditions, such as Alzheimer's disease and Parkinson's disease, affect mainly older people. Some neurological conditions have a sudden onset due to injury or illness, such as a head injury or stroke, or cancers of the brain and spine.

In an embodiment, the neurological condition is the result of traumatic damage to the brain. Additionally or alternatively, the neurological condition is the result of an energy deficiency in the brain or in the muscles.

Examples of neurological conditions include migraine, memory disorder, age-related memory disorder, brain injury, neurorehabilitation, stroke and post-stroke, amyloid lateral sclerosis, multiple sclerosis, cognitive impairment, mild cognitive impairment (MCI), cognitive impairment post-intensive care, age-induced cognition impairment, Alzheimer's disease, Parkinson's disease, Huntingdon's disease, inherited metabolic disorders (such as glucose transporter type 1 deficiency syndrome and pyruvate dehydrogenase complex deficiency), bipolar disorder, schizophrenia, and/or epilepsy.

A migraine is an intense headache accompanied by other symptoms such as nausea (feeling sick), visual problems and an increased sensitivity to light or sound. A migraine may be preceded by an aura; the main symptoms of an aura are visual problems such as blurred vision (difficulty focusing), blind spots, flashes of light, or a zigzag pattern moving from the central field of vision towards the edge.

Strokes (also known as cerebrovascular accident (CVA) and cerebrovascular insult (CVI)) occur when there is poor blood flow to the brain resulting in cell death. There are two main types of stroke: ischemic (due to lack of blood flow) and haemorrhagic (due to bleeding). Strokes result in part of the brain not functioning properly. The signs and symptoms of a stroke may include an inability to move or feel on one side of the body, problems understanding or speaking, feeling like the world is spinning, or loss of vision to one side. The signs and symptoms often appear soon after the stroke has occurred.

Amyotrophic lateral sclerosis (ALS) (also known as Lou Gehrig's disease, Charcot disease and motor neuron disease), involves the death of neurons responsible for controlling voluntary muscles. ALS is characterized by stiff muscles, muscle twitching, and gradually worsening weakness due to muscle wasting; this results in difficulty speaking, swallowing, and eventually breathing.

Multiple sclerosis affects the nerves in the brain and spinal cord, causing a wide range of symptoms including problems with muscle movement, problems with mobility and balance, numbness and tingling, blurring of vision (typically there is loss of vision in one eye) and fatigue.

Parkinson's disease is a degenerative disorder of the central nervous system mainly affecting the motor system. In the early course of the disease, the most obvious symptoms are movement-related; these include tremor at rest, rigidity, slowness of movement and difficulty with walking and gait. Later in the course of the disease, thinking and behavioral problems may arise, with dementia commonly occurring in the advanced stages of the disease. Other symptoms include depression, sensory, sleep and emotional problems.

Alzheimer's disease is a progressive neurodegenerative disorder. Alzheimer's disease is the most common cause of dementia. Symptoms include memory loss and difficulties with thinking, problem-solving or language. The mini mental state examination (MMSE) is an example of one of the tests used to diagnose Alzheimer's disease.

Huntington's disease is an inherited condition that damages certain nerve cells in the brain. Huntington's disease affects muscle coordination and leads to mental decline and behavioral symptoms. The earliest symptoms are often subtle problems with mood or cognition. A general lack of coordination and an unsteady gait often follow. As the disease advances, uncoordinated, jerky body movements become more apparent, along with a decline in mental abilities and behavioral symptoms. Physical abilities gradually worsen until coordinated movement becomes difficult. Mental abilities generally decline into dementia.

Inherited metabolic disorders are a range of diseases caused by defective genes. Typically the defective gene(s) results in a defect in an enzyme or in a transport protein which results in a block in the way that a compound is processed by the body such that there is a toxic accumulation of the compound. Inherited metabolic disorders can affect any organ and usually affect more than one. Symptoms often tend to be nonspecific and usually relate to major organ dysfunction or failure. The onset and severity of a metabolic disorder may be exacerbated by environmental factors, such as diet and concurrent illness.

Glucose transporter type 1 (Glut1) deficiency syndrome is a genetic metabolic disorder involving the GLUT1 protein which transports glucose across the blood-brain barrier or the boundary separating tiny blood vessels from brain tissue. The most common symptom is seizures (epilepsy), which usually begin within the first few months of life. Additional symptoms that can occur include varying degrees of cognitive impairment and movement disorders characterized by ataxia, dystonia, and chorea. Glut1 deficiency syndrome may be caused by mutations in the SLC2A1 gene which produce GLUT1 protein.

Pyruvate dehydrogenase complex deficiency (pyruvate dehydrogenase deficiency or PDCD) is a neurodegenerative disorder associated with abnormal mitochondrial metabolism and disrupted carbohydrate metabolism. PDCD is characterized by the buildup of lactic acid in the body and a variety of neurological problems. Signs and symptoms of this condition usually first appear shortly after birth, and they can vary widely among affected individuals. The most common feature is a potentially life-threatening buildup of lactic acid (lactic acidosis), which can cause nausea, vomiting, severe breathing problems, and an abnormal heartbeat. Other symptoms include: neurological problems; delayed development of mental abilities and motor skills such as sitting and walking; intellectual disability; seizures; weak muscle tone (hypotonia); poor coordination, and difficulty walking. Some affected individuals have abnormal brain structures, such as underdevelopment of the tissue connecting the left and right halves of the brain (corpus callosum), wasting away (atrophy) of the exterior part of the brain known as the cerebral cortex, or patches of damaged tissue (lesions) on some parts of the brain.

PDCD is a deficiency of one of the proteins in the pyruvate dehydrogenase complex (PDC). The pyruvate dehydrogenase complex comprises three enzymes identified as E1, E2 and E3; the E1 enzyme contains subunits identified as alpha and beta. The most common form of PDCD is caused by an abnormal gene in the E1 alpha subunit (the PDHA1 gene) located on the X chromosome. Some PDCD cases are caused by a mutation in a gene in another subunit of the pyruvate dehydrogenase complex such as the PDHX gene, the PDHB gene, the DLAT gene, the PDP1 gene, and the DLD gene.

Bipolar disorder is a brain disorder that causes unusual shifts in mood, energy, activity levels, and the ability to carry out day-to-day tasks. Bipolar disorder is characterized by periods of elevated mood and periods of depression. Bipolar disorder can be diagnosed using the guidelines from the Diagnostic and Statistical Manual of Mental Disorders (DSM) or the World Health Organization's International Statistical Classification of Diseases and Related Health Problems.

Schizophrenia is a chronic, severe, and disabling brain disorder in which individuals interpret reality abnormally. Schizophrenia may result in some combination of hallucinations, hearing voices, delusions, and extremely disordered thinking and behavior. Schizophrenia can be diagnosed using the guidelines from the Diagnostic and Statistical Manual of Mental Disorders (DSM) or the World Health Organization's International Statistical Classification of Diseases and Related Health Problems.

Epilepsy is a neurological disorder in which nerve cell activity in the brain becomes disrupted, causing seizures or periods of unusual behavior, sensations and sometimes loss of consciousness.

The terms “cognitive impairment” and “cognition impairment” refer to disorders that give rise to impaired cognition, in particular disorders that primarily affect learning, memory, perception, and/or problem solving.

Cognitive impairment may occur in a subject after intensive care. Cognitive impairment may occur as part of the ageing process, e.g. mild cognitive impairment (MCI).

The term “cognition” refers to the set of all mental abilities and processes, including knowledge, attention, memory and working memory, judgment and evaluation, reasoning and “computation”, problem solving and decision making, comprehension and production of language. Levels of and improvements in cognition can be readily assessed by the skilled person using any suitable neurological and cognitive tests that are known in the art, including cognitive tests designed to assess speed of information processing, executive function and memory. Suitable example tests include Mini Mental State Examination (MMSE), Cambridge Neuropsychological Test Automated Battery (CANTAB), Alzheimer's Disease Assessment Scale-cognitive test (ADAScog), Wisconsin Card Sorting Test, Verbal and Figural Fluency Test and Trail Making Test, Wechsler Memory scale (WIVIS), immediate and delayed Visual Reproduction Test (Trahan et al. Neuropsychology, 1988 19(3) p. 173-89), the Rey Auditory Verbal Learning Test (RAVLT) (Ivnik, R J. et al. Psychological Assessment: A Journal of Consulting and Clinical Psychology, 1990 (2): p. 304-312), electroencephalography (EEG), magnetoencephalography (MEG), Positron Emission Tomography (PET), Single Photon Emission Computed Tomography (SPECT), Magnetic Resonance Imaging (MRI), functional Magnetic Resonance Imaging (fMRI), computerized tomography and long-term potentiation.

EEG, a measure of electrical activity of the brain, is accomplished by placing electrodes on the scalp at various landmarks and recording greatly amplified brain signals. MEG is similar to EEG in that it measures the magnetic fields that are linked to electrical fields. MEG is used to measure spontaneous brain activity, including synchronous waves in the nervous system.

PET provides a measure of oxygen utilization and/or glucose metabolism. In this technique, a radioactive positron-emitting tracer is administered, and tracer uptake by the brain is correlated with brain activity. These tracers emit gamma rays which are detected by sensors surrounding the head, resulting in a 3D map of brain activation. As soon as the tracer is taken up by the brain, the detected radioactivity occurs as a function of regional cerebral blood flow. During activation, an increase in cerebral blood flow and neuronal glucose metabolism can be detected within seconds.

Suitable analysis can also be based on neuropsychiatric testing, clinical examinations and individual complaints of loss of cognitive function (e.g. subjective memory loss). Further suitable tests may be based on assessments of locomotion, memory and attention, seizure susceptibility, and social interaction and/or recognition.

Memory disorders are the result of neurological damage to the brain structures such that the storage, retention and recollection of memories are hindered. Memory disorders can be progressive with age (e.g. Alzheimer's disease), or they can be immediately resulting, for example, from a head injury. Levels of and improvements in memory disorders can be readily assessed by the skilled person using any suitable tests that are known in the art such as Alzheimer's Disease Assessment Scale-cognitive test (ADAScog), Mini Mental State Examination (MMSE), computerized tomography (CT) scan, Magnetic Resonance Imaging (MRI), Single Photon Emission Computed Tomography (SPECT), Positron Emission Tomography (PET), and electroencephalography (EEG).

### EXAMPLES

The following non-limiting examples present scientific data developing and supporting the concept of an MCT based composition with improved sedimentation and mouthfeel attributes comprising specific gum(s), as provided by the present disclosure.

In the examples, physical stability of the samples was observed and assessed. As used herein, the physical stability means the inherent tendency of emulsions and suspensions or mixed systems to separate into distinct phases. If instability has progressed in a dairy or nutritional product, different phases become visible such as creaming, serum and sediment. It can be reversible or irreversible. The physical stability of the samples was assessed by creaming, serum, and sediment, each of which is rated on a scale of 0 to 5, 0 being no evidence and 5 being the highest level/severity.

Creaming is the floating of fat particles (droplets or solids) to the surface of a product. Creaming can be of cloudy nature, i.e. slight fat separation is visible as very fine streaks on the product surface. Creaming can occur in a layer, i.e. the fat covers the product surface or occurs as lumps or specks, i.e. separated fat forms irreversible lumps or specks on the product surface or the lid. Creaming is evaluated based on the amount of fat deposit on the product surface and on the lid of the package. The ratings of creaming are as follows:

### Description

0 Zero=no creaming is visible
1 Trace=lid or product surface is a quarter covered with thin layer of fat deposit
2 Slight=lid or product surface is half covered with thin layer of fat deposit
3 Distinct=lid or product surface is 3 quarters covered with thin layer of fat deposit (<1 mm)
4 Heavy=lid or product surface is fully covered with a layer of fat deposit (1-2 mm)
5 Very heavy=lid or product surface is fully covered with thick layer of fat deposit (>2 mm)

Serum may be formed if liquid dairy and nutritional products show creaming and/or sedimentation. It can also appear if a network (proteins and/or hydrocolloids) exhibits syneresis. Serum may be clear or turbid. Serum is evaluated by pouring the liquid into a glass beaker. Different scores of evaluation of serum are as follows:

| Score | Description |
|---|---|
| 0 | Zero = no serum is visible |
| 1 | Trace = trace of a line during pouring |
| 2 | Slight = ~2% of packaging volume |
| 3 | Distinct = ~5% of packaging volume |
| 4 | Heavy = Heavy = ~10% of packaging volume |
| 5 | Very heavy =≥ 20% of packaging volume |

Sedimentation is the settling of particles at the bottom of the package. The sediment is evaluated at the bottom of the package after pouring the liquid into a glass beaker.

| Score | Description |
|---|---|
| 0 | Zero = no sediment visible on the bottom |
| 1 | Trace = the bottom is covered near the walls |
| 2 | Slight = ~1 mm middle seal is still clearly visible |
| 3 | Distinct = ~2 mm middle seal is just visible |
| 4 | Heavy = ~3 mm (assess with ruler) |
| 5 | Very heavy =≥ 4 mm (assess with ruler) |

All the samples below include a 15 g dose of MCT per serving size and 5% MPC or 7.5% MPC.

### Example 1: 7.5% MPC (Control 1)

| | Temperature | | | | Sediment |
|---|---|---|---|---|---|
| Time | (Celsius) | Creaming | Serum | Sediment | (Cm) |
| 2 week | ambient | 1 | 0 | 4 | 2.4 |
| 2 week | fridge | 2 | 0 | 4 | 2.6 cm |
| | | | | | before |
| | | | | | and 1.5 |
| | | | | | cm after |
| 2 week | 35 | 2 | 0 | 4 | 2.5 cm |
| 2 week | 45 | 2 | 0 | 4 | |

### Example 2: Gellan Gum (Control 2)+5% MPC

| | | Temperature | | | | Layer | | |
|---|---|---|---|---|---|---|---|---|
| Time | | (Celsius) | Creaming | Serum | Sediment | On Top | Sediment (Cm) | Comment |
| 2 | week | ambient | 1 | 0 | 1 | | | very slight |
| | | | | | | | | residual/sediment |
| 2 | week | fridge | 1 | 0 | 0 | | | |
| 2 | week | 35 | 0 | 0 | 1 | | | slight residual |
| 2 | week | 45 | 2 | 5 | 3 | YES | | loose protein on |
| | | | | | | | | bottom |
| 1 | month | ambient | 0 | 0 | 0 | | | more bottle |
| | | | | | | | | coating than with |
| | | | | | | | | carageenan |
| 1 | month | fridge | 0 | 0 | 0 | | | |
| 1 | month | 35 | 1 | 1 | 4 | | 1 cm on | creaming layer |
| | | | | | | | bottom | on top |
| 1 | month | 45 | 2 | 5 | | | | |

### Example 3: Gellan Gum+Iota Caraeenan+7.5% MPC

| | | Temperature | | | | | | |
|---|---|---|---|---|---|---|---|---|
| Time | | (Celsius) | Creaming | Serum | Sediment | Rippliing | Sediment (Cm) | Comment |
| 2 | week | ambient | 0 | 0 | 0 | | | |
| 2 | week | fridge | 0 | 0 | 0 | | | |
| 2 | week | 35 | 0 | 0 | 0 | | | |
| 2 | week | 45 | 0 | 1 | 3 | yes | 7 nun | no visual |
| | | | | | | (serum) | after pour | separation |
| | | | | | | | | beforehand |
| 1 | month | ambient | 0 | 0 | 0 | | | some coating on |
| | | | | | | | | bottles (sticky |
| | | | | | | | | milk but no |
| | | | | | | | | sediment) |
| 1 | month | fridge | 0 | 0 | 0 | | | same comment as |
| | | | | | | | | above |
| 1 | month | 35 | 0 | 0 | 0 | | | |
| 1 | month | 45 | 2 | 3 | 4 | | | second half of |
| | | | | | | | | pour was discolored |
| | | | | | | | | when pouring . . . |
| | | | | | | | | protein somewhat |
| | | | | | | | | shook in with |
| | | | | | | | | gentle pouring back |
| | | | | | | | | and forth and with |
| | | | | | | | | shaking, went |
| | | | | | | | | back in |
| 2 | month | ambient | 0 | 0 | 0 | | | looks great |
| 2 | month | fridge | 0 | 0 | 0 | | | looks great |
| 2 | month | 35 | 0 | 0 | 1 | | | looks pretty good |
| | | | | | | | | unshaken had slight |
| | | | | | | | | residual on bottom |
| 2 | month | 45 | | | | | | way too cooked ignore |
| 3 | month | ambient | 0 | 0 | 0 | | | ½ mm top ‘cream’ - |
| | | | | | | | | low issue |
| 3 | month | fridge | 0 | 0 | 0 | | | ½ mm top ‘cream’ - |
| | | | | | | | | low issue |

### Example 4: Cellulose Gel/Gum (MCC 85%/15% CMC) High Viscosity+MPC

| | | Temperature | | | | |
|---|---|---|---|---|---|---|
| Time | | (Celsius) | Creaming | Serum | Sediment | Comment |
| 2 | week | ambient | 0 | 0 | 0 | |
| 2 | week | fridge | 0 | 0 | 0 | |
| 2 | week | 35 | 0 | 0 | 0 | |
| 2 | week | 45 | 3 | 0-1 | 1 | creaming was heavy |
| | | | | | | complete ring . . . |
| | | | | | | sedimentation |
| | | | | | | very minor |
| 1 | month | ambient | 0 | 0 | 0 | |
| 1 | month | fridge | 0 | 0 | 0 | |
| 1 | month | 35 | 0 | 0 | 0 | some coating on bottle |
| | | | | | | from high protein |
| 1 | month | 45 | 0 | 0 | 0 | |
| 2 | month | ambient | 0 | 0 | 0 | very slight residual - |
| | | | | | | maybe even a zero |
| 2 | month | fridge | 0 | 0 | 0 | |
| 2 | month | 35 | 0 | 0 | 1 | |
| 2 | month | 45 | | | | |
| 3 | month | ambient | 0 | 0 | 0 | |

### Example 5: Cellulose Gel/Gum (MCC 85%/15% CMC) High Viscosity+5% MPC

| | | Temperature | | | | | | |
|---|---|---|---|---|---|---|---|---|
| Time | | (Celsius) | Creaming | Serum | Sediment | Rippliing | Sediment (Cm) | Comment |
| 2 | week | ambient | 0 | 0 | 2 | slight | 4 mm after | none visually |
| | | | | | | | pour edges | |
| 2 | week | fridge | 0 | 0 | 1 | no | | none visually |
| 2 | week | 35 | 0 | 0 | 1 Jan. | yes slight | 2 mm on | none visually |
| | | | | | | | average edges | |
| 2 | week | 45 | 2 | 0 | 2 | | 5 mm just | none visually |
| | | | | | | | around edges | |
| 1 | month | ambient | 0 | 2 | 2 | slight | 4 mm around | none visually - |
| | | | | | | | the edges | mixes in |
| 1 | month | fridge | 0 | 1 | 1 | yes slight | | none visually - |
| | | | | | | | | mixes in |
| 1 | month | 35 | 1 | 2 | 2 | yes | 4 mm around | 2 cm top |
| | | | | | | | the edges | ‘serum’ - |
| | | | | | | | | mixes in |
| 1 | month | 45 | 0 | 3 | 2 | yes | 4 mm around | 4 cm top |
| | | | | | | | the edges | ‘serum’ - |
| | | | | | | | | mixes in |
| 2 | month | ambient | | | | | | |
| 2 | month | fridge | 1 | 1 | 1 | slight | | |
| 3 | month | ambient | 1 | 2 | 2 | | | |
| 3 | month | fridge | 1 | 1 | 2 | | | |
| 3 | month | 35 | 1 | 2 | 2 | | | |

### Example 6: Gellan Gum+Iota Carageenan+5% MPC

| | | Temperature | | | | | |
|---|---|---|---|---|---|---|---|
| Time | | (Celsius) | Creaming | Serum | Sediment | Sediment (Cm) | Comment |
| 2 | week | ambient | 0 | 0 | 0 | | no visual separation - |
| | | | | | | | looks perfect . . . |
| 2 | week | fridge | 0 | 0 | 0 | | no visual separation- |
| | | | | | | | looks perfect . . . |
| 2 | week | 35 | 0 | 0 | 0 | | no visual separation - |
| | | | | | | | looks great |
| 2 | week | 45 | 1 | 0 | 2 | 4 mm on edges - | no visual separation - |
| | | | | | | loose - will | |
| | | | | | | shake in | |
| 1 | month | ambient | 0 | 0 | 0 | | |
| 1 | month | fridge | 0 | 0 | 0 | | |
| 1 | month | 35 | 0 | 0 | 0 | | |
| 1 | month | 45 | 2 | 0 | 2 | | |
| 2 | month | ambient | 0 | 0 | 0 | | |
| 2 | month | fridge | 1 | 0 | 0 | | very faint creaming |
| | | | | | | | ring |
| 3 | month | ambient | 0 | 0 | 0 | | |
| 4 | month | ambient | 0 | 0 | 0 | | still looks perfect |
| | | | | | | | (after pouring |
| | | | | | | | coating throughout |
| | | | | | | | bottle) |

### Example 7: 5% MPC+Gellan Gum+Iota Carageenan

| | | Temperature | | | | | | |
|---|---|---|---|---|---|---|---|---|
| Time | | (Celsius) | pH | Viscosity | Creaming | Serum | Sediment | Comment |
| 1 | month | ambient | 6.88 | 23.8 | 0 | 0 | 0 | |
| 1 | month | fridge | 6.92 | 41.3 | 0 | 0 | 0 | |
| 1 | month | 35 | 6.87 | 22 | 0 | 1 | 0 | can see color change |
| | | | | | | | | ⅓ up bottle but can |
| | | | | | | | | easily shake in serum |
| 1 | month | 45 | 6.8 | 17.6 | 0 | 1 | 0 | can see color change |
| | | | | | | | | ⅓ up bottle but can |
| | | | | | | | | easily shake in serum |
| 2 | month | ambient | 6.9 | 23.5 | 0 | 0 | 1 | slight rippling so 1 |
| | | | | | | | | for serum |
| 2 | month | 35 | 6.87 | 20.2 | 0 | 2 | 1 | slight ring sediment . . . |
| | | | | | | | | serum shook in |
| 3 | month | ambient | 6.86 | 21.9 | 0 | 0 | 0 | |
| 3 | month fridge | fridge | 6.91 | 39.3 | 0 | 0 | 0 | |
| 3 | month | 35 | 6.8 | 18.7 | 0 | 1 | 1 | swirling when |
| | | | | | | | | pouring - darker |
| | | | | | | | | brown color at end |
| | | | | | | | | and parts were clear |
| 9 | month | ambient | 6.77 | 19 | 2 | 3 | 2 | |
| 9 | month | fridge | 6.85 | 27.8 | 0 | 0 | 0 | |

### Example 8: 7.5% MPC+Cellulose Gel/Gum (MCC 85%/15% CMC)

| | Temperature | | | | | | |
|---|---|---|---|---|---|---|---|
| Time | (Celsius) | pH | Viscosity | Creaming | Serum | Sediment | Comment |
| 1 month | ambient | 6.81 | 34.1 | 0 | 0 | 0 | |
| 1 month | fridge | 6.88 | 43.2 | 0 | 0 | 0 | |
| 1 month | 35 | 6.77 | 34.7 | 0 | 0 | 0 | |
| 1 month | 45 | 6.7 | 35.6 | 3 | 0 | 0 | Creaming could be |
| | | | | | | | from bubbles in |
| | | | | | | | bottle (not really |
| | | | | | | | with time) |
| 2 month | ambient | 6.82 | 41.7 | 0 | 0 | 0 | |
| 2 month | 35 | 6.8 | 41.7 | 1 | 0 | 0 | |
| 2 month | 45 | 6.71 | 38.5 | 3 | 0 | 0 | |
| 3 month | ambient | 6.77 | 37.8 | 0 | 0 | 0 | Bottle coating . . . |
| | | | | | | | some visible oil |
| | | | | | | | droplets on top of |
| | | | | | | | formulation (I saw |
| | | | | | | | this in US MCT oil |
| | | | | | | | products too . . . ) |
| 3 month | fridge | 6.81 | 48.6 | 0 | 0 | 0 | “ slightly less |
| | | | | | | | bottle coating |
| 3 month | 35 | 6.75 | 35.9 | 1 | 0 | 0 | |
| 6 month | ambient | 6.74 | 43.9 | 1 | 0 | 0 | |
| 6 month | fridge | 6.8 | 71.8 | 0 | 0 | 0 | |
| 9 month | ambient | 6.72 | 42.8 | 1 | 0 | 0 | |
| 9 month | fridge | 6.74 | 62.4 | 0 | 0 | 0 | |

### Example 9: 7.5% MPC+Gellan Gum+Iota Carageenan

| | Temperature | | | | | | |
|---|---|---|---|---|---|---|---|
| Time | (Celsius) | pH | Viscosity | Creaming | Serum | Sediment | Comment |
| 1 month | ambient | 6.81 | 42.1 | 0 | 0 | 0 | |
| 1 month | fridge | 6.85 | 61.1 | 0 | 0 | 0 | |
| 1 month | 35 | 6.77 | 31 | 0 | 1 | 1 | can see color change |
| | | | | | | | ⅓ up bottle but can |
| | | | | | | | easily shake in serum, |
| | | | | | | | some sediment (thin ring) |
| 1 month | 45 | 6.71 | 29.5 | 0 | 1 | 3 | can see color change |
| | | | | | | | ⅓ up bottle but can |
| | | | | | | | easily shake in serum, |
| | | | | | | | thin layer sediment on |
| | | | | | | | bottom (darker color) |
| 2 month | ambient | 6.83 | 43.7 | 1 | 0 | 0 | |
| 2 month | 35 | 6.81 | 29 | 1 | 1 | 3 | |
| 2 month | 45 | 6.75 | n/a | 4 | 2 | 4 | |
| 3 month | ambient | 6.78 | 41.5 | 0 | 0 | 0 | |
| 3 month | fridge | 6.88 | 60.5 | 3 | 0 | 0 | |
| 3 month | 35 | 6.8 | 31.3 | 3 | 2 | 4 | large protein pad |
| | | | | | | | on bottom . . . |
| 6 month | ambient | 6.81 | 36.8 | 2 | 0 | 1 | sediment loose ring |
| 6 month | fridge | 6.86 | 63.9 | 2 | 0 | 1 | very very light |
| | | | | | | | sediment ring |
| 9 month | ambient | 6.71 | 32.5 | 3 | 0 | 3 | |
| 9 month | fridge | 6.76 | 56 | 3 | 0 | 0 | |

### Example 10: 5% MPC+Cellulose Gel/Gum (MCC 85%/15% CMC)

| | Temperature | | | | |
|---|---|---|---|---|---|
| Time | (Celsius) | Creaming | Serum | Sediment | Comment |
| 1 month | ambient | 2 | 0 | 0 | faint ring |
| | | | | | creaming |
| 1 month | fridge | 0 | 0 | 0 | |
| 1 month | 35 | 1 | 0 | 0 | faint |
| | | | | | creaming |
| 1 month | 45 | 0 | 0 | 0 | |
| 2 month | ambient | 0 | 0 | 0 | |
| 2 month | 35 | 1 | 0 | 0 | |
| 2 month | 45 | 3 | 0 | 0 | |
| 3 month | ambient | 1 | 0 | 0 | light coating |
| | | | | | on bottle but |
| | | | | | typical to |
| | | | | | Benefic |
| 3 month | fridge | 0 | 0 | 0 | |
| 3 month | 35 | 2 | 0 | 0 | |
| 6 month | ambient | 0 | 0 | 0 | |
| 6 month | fridge | 0 | 0 | 0 | |
| 9 month | ambient | missing | missing | missing | |
| 9 month | fridge | 3 | 0 | 0 | |

### Example 11: 7.5% MPC+Cellulose Gel/gum (MCC 85%/15% CMC)

| | Temperature | | | | | | |
|---|---|---|---|---|---|---|---|
| Time | (Celsius) | pH | Viscosity | Creaming | Serum | Sediment | Comment |
| 1 month | ambient | 6.76 | 31.9 | 2 | 0 | 0 | faint ring |
| | | | | | | | creaming |
| 1 month | fridge | 6.91 | 37.3 | 0 | 0 | 0 | |
| 1 month | 35 | 6.86 | 33.3 | 1 | 0 | 0 | faint creaming |
| 1 month | 45 | 6.79 | 35.6 | ( ) | 0 | 0 | |
| 2 month | ambient | 6.9 | 41.4 | 0 | 0 | 0 | |
| 2 month | 35 | 6.86 | 40.5 | 1 | 0 | 0 | |
| 2 month | 45 | 6.78 | n/a | 3 | 0 | 0 | |
| 3 month | ambient | 6.89 | 39.8 | 1 | 0 | 0 | light coating on |
| | | | | | | | bottle but typical |
| | | | | | | | to Benefic |
| 3 month | fridge | 6.93 | 43.4 | 0 | 0 | 0 | |
| 3 month | 35 | 6.87 | 41.6 | 2 | 0 | 0 | |
| 6 month | ambient | 6.83 | 45.5 | 0 | 0 | 0 | |
| 6 month | fridge | 6.88 | 44.6 | 0 | 0 | 0 | |
| 9 month | ambient | 6.74 | 40.6 | missing | missing | missing | |
| 9 month | fridge | 6.83 | 38.7 | 3 | 0 | 0 | |

### Example 12: 7.5% MPC+Cellulose Gel/Gum (MCC 85%/15% CMC)+Cappuccino Flavor

| | Temperature | | | | | | |
|---|---|---|---|---|---|---|---|
| Time | (Celsius) | pH | Viscosity | Creaming | Serum | Sediment | Comment |
| 1 month | ambient | 6.71 | 36.2 | 1 | 0 | 0 | |
| 1 month | fridge | 6.75 | 33.3 | 0 | 0 | 0 | |
| 1 month | 35 | 6.7 | 32.4 | 1 | 0 | 2 | some rippling |
| | | | | | | | (marbled), very |
| | | | | | | | loose light ring |
| | | | | | | | sediment |
| 1 month | 45 | 6.62 | 38.8 | 4 | 0 | 2 | some rippling |
| | | | | | | | (marbled), sediment |
| | | | | | | | light loose ring, |
| | | | | | | | bubbly creaming ring |
| 3 month | ambient | 6.66 | 35.1 | 0 | 0 | 0 | |
| 3 month | fridge | 6.71 | 36.6 | 0 | 0 | 0 | |
| 3 month | 35 | 6.62 | 32.2 | 0 | 4 | 3 | after shaking, |
| | | | | | | | serum and sediment 0 |

### Example 13: 7.5% MPC+Cellulose Gel/Gum (MCC 85%/15% CMC)+Vanilla Chai Flavor

| | Temper- | | | | |
|---|---|---|---|---|---|
| | ature | | | | |
| Time | (Celsius) | Creaming | Serum | Sediment | Comment |
| 1 month | ambient | 3 | 0 | 1 | one spot heavy |
| | | | | | creaming, |
| | | | | | sediment |
| | | | | | was loose |
| | | | | | light ring, |
| | | | | | cinnamon |
| | | | | | specks |
| | | | | | minor on |
| | | | | | bottom |
| 1 month | fridge | 0 | 0 | 0 | |
| 1 month | 35 | 1 | 0 | 3 | 1 mm bottom |
| | | | | | coverage |
| | | | | | mixes in |
| 1 month | 45 | 4 | 0 | 3 | creaming ring |
| | | | | | from bubbles, |
| | | | | | 1 mm bottom |
| | | | | | coverage |
| 3 month | ambient | 0 | 2 | 1 | |
| 3 month | fridge | 0 | 1 | 0 | |
| 3 month | 35 | 0 | 4 | 2 | sediment 1 |
| | | | | | after shaking, |
| | | | | | slight loose |
| | | | | | chunks when |
| | | | | | pouring |

### Example 14: 7.5% MPC+Cocoa Unflavored

| | Temper- | | | | |
|---|---|---|---|---|---|
| | ature | | | | |
| Time | (Celsius) | Creaming | Serum | Sediment | Comment |
| 1 month | ambient | 1 | 0 | 0 | slight bubble |
| | | | | | creaming |
| 1 month | fridge | 0 | 0 | 0 | |
| 1 month | 35 | 3 | 0 | 2 | bubbly ring, |
| | | | | | scrum rippling |
| | | | | | mixed in, |
| | | | | | sediment ring |
| | | | | | loose with |
| | | | | | specks |
| 1 month | 45 | 1 | 0 | 0 | very light |
| | | | | | loose ring |
| | | | | | sediment, |
| | | | | | some bubbly |
| | | | | | creaming |
| 3 month | ambient | 1 | 1 | 0 | |
| 3 month | fridge | 0 | 0 | 0 | |
| 3 month | 35 | 0 | 5 | 2 | cocoa |
| | | | | | separation |
| | | | | | when |
| | | | | | flouring- |
| | | | | | easily |
| | | | | | shakes in |

### Example 15: 7.5% MPC+Cellulose Gel/Gum (MCC 85%/15% CMC)+Mocha Flavor

| | Temper- | | | | |
|---|---|---|---|---|---|
| | ature | | | | |
| Time | (Celsius) | Creaming | Serum | Sediment | Comment |
| 1 month | ambient | 1 | 0 | 0 | very light |
| | | | | | sediment ring, |
| | | | | | some bubbly |
| | | | | | creaming |
| 1 month | fridge | 0 | 0 | 0 | |
| 1 month | 35 | 0 | 0 | 2 | serum, ripping |
| | | | | | mixed in, |
| | | | | | sediment |
| | | | | | slight ring |
| 1 month | 45 | 0 | 0 | 3 | loose |
| | | | | | coverage |
| | | | | | on bottom |
| | | | | | with cocoa |
| | | | | | specks, easily |
| | | | | | mixes in |
| 3 month | ambient | 2 | 1 | 0 | creaming in |
| | | | | | one spot |
| 3 month | fridge | 0 | 0 | 0 | |
| 3 month | 35 | 0 | 5 | 2 | cocoa |
| | | | | | separation |
| | | | | | when |
| | | | | | pouring- |
| | | | | | easily |
| | | | | | shakes in |

Samples of this composition without pH adjustment (labeled 1-2, pH=6.55) and with pH adjustment by KOH (labeled 3-6, pH=6.79 and 6.99). Samples Nos. 3-6 were placed in hot water bath for different time periods, and no fouling, coagulation, or aggregation was observed. However, sample Nos. 1-2 with pH of 6.55 showed gelling/fouling. Overall, adjusting the pH to 6.8-7.0 shows more stability in processing, and this minor pH change does not have huge impacts on finished product.

### Example 16: 7.5% MPC+Cellulose Gel/Gum (MCC 85%/15% CMC)+Pineapple Mango Flavor

| | Temper- | | | | |
|---|---|---|---|---|---|
| | ature | | | | |
| Time | (Celsius) | Creaming | Serum | Sediment | Comment |
| 1 month | ambient | 2 | 0 | 1 | creaming, |
| | | | | | partial ring |
| | | | | | but thick . . . |
| | | | | | sediment |
| | | | | | light ring |
| 1 month | fridge | 0 | 0 | 0 | |
| 1 month | 35 | 0 | 0 | 2 | thicker ring |
| | | | | | sediment |
| 1 month | 45 | 0 | 0 | 3 | sediment |
| | | | | | covered ¾th |
| | | | | | of bottom |
| 3 month | ambient | 3 | 2 | 1 | not full |
| | | | | | creaming ring |
| 3 month | fridge | 0 | 4 | 2 | sediment loose |
| | | | | | ring, no |
| | | | | | creaming ring, |
| | | | | | sediment light, |
| | | | | | shakes |
| | | | | | in fine |
| 3 month | 35 | 0 | 4 | 2 | sediment |
| | | | | | loose ring- |
| | | | | | shakes in fine |

### Example 17: 0.5% Cellulose Gel/Gum (MCC 85%/15% CMC)+5% MPC

| | Temperature | | | |
|---|---|---|---|---|
| Time | (Celsius) | Creaming | Serum | Sediment |
| 2 week | ambient | 0 | 1 | 1 |
| 2 week | fridge | 0 | 0 | 0 |
| 2 week | 35 | 0 | 1 | 1 |
| 2 week | 45 | 3 | 0 | 1 |
| 1 month | ambient | 0 | 2 | 1 |
| 1 month | fridge | 0 | 0 | 0 |
| 1 month | 35 | 0 | 1 | 0 |
| 1 month | 45 | 2 | 0 | 1 |

### Example 18: Iota Carageenan (Control 3)+7.5% MPC

| | Temperature | | | | | |
|---|---|---|---|---|---|---|
| Time | (Celsius) | Creaming | Serum | Sediment | Sediment (cm) | Comment |
| 2 week | ambient | 0 | 0 | 5 | 3.2 cm on | |
| | | | | | bottom | |
| 2 week | fridge | 0 | 0 | 3 | | loose sediment |
| | | | | | | that shook back in |
| 2 week | 35 | 3 | 0 | 5 | 2.8 cm on | |
| | | | | | bottom | |
| 2 week | 45 | 2 | 0 | 4 | 2.5 cm on | |
| | | | | | bottom | |

The following tables include more details of the above Examples:

| | | | Total | Viscosity | Total | | | Sugar |
|---|---|---|---|---|---|---|---|---|
| Ex # | Description | pH | Protein | (cps) | Solids | Density | Total Fat | Profile |
| 4 | Cellulose gel/gum | 6.9 | 5.95 | 18.5 | 19.34 | | | |
| | (MCC 85%/15% | | | | | | | |
| | CMC)High Viscosity | | | | | | | |
| 5 | Cellulose gel/gum | 6.82 | | 14.5 | 16.77 | | | |
| | (MCC 85%/15% CMC) | | | | | | | |
| | High Viscosity | | | | | | | |
| | 5% MPC | | | | | | | |
| 6 | Gellan Gum & Iota | 6.83 | | 20.1 | 16.02 | | | |
| | Carageenan 5% MPC | | | | | | | |
| 13 | 5% MPC - unflavored | 6.96 | 4.03% | 12.8 | 17.68 | | 11.90% | |
| | with Cellulose gel/gum | | | | | | | |
| | (MCC 85%/15% CMC) | | | | | | | |
| 7 | 5% MPC, Gellan | | 4.20 g/100 g | | 18.31 | 1 | 12.24 g/100 g | 1.48 g/100 g |
| | gum, Iota caraueenan | | | | | | | |
| | beginning nutritionals | 7.02 | | 39 | 19.34 | 1.011 | | |
| | middle nutritionals | 7.02 | | 38.8 | 19.31 | 1.011 | | |
| | end nutritionals | 7.01 | | 37 | 19.06 | 1.011 | | |
| 8 | 7.5% MPC, Cellulose | | 6.30 g/100 g | | 21 | 1 | 12.24 g/100 g | 1.46 g/100 g |
| | gel/gum (MCC | | | | | | | |
| | 85%/15% CMC) | | | | | | | |
| | beginning nutritionals | 6.85 | | 32.1 | 22.15 | 1.02 | | |
| | middle nutritionals | 6.85 | | 30.9 | 22.06 | 1.02 | | |
| | end nutritionals | 6.87 | | 31.4 | 22 | | | |
| 9 | 7.5% MPC. Gellan | | 6.30 g/100 g | | 19.47 | | 12.24 g/100 g | 0.38 g/100 g |
| | gum, Iota carageenan | | | | | | | |
| | beginning nutritionals | 6.89 | | 50.88 | 19.99 | 1.013 | | |
| | middle nutritionals | 6.92 | | 50.7 | 20.02 | 1.013 | | |
| | end nutritionals | 6.92 | | 52.2 | 20.02 | 1.013 | | |

| | | | | | pH | | | |
|---|---|---|---|---|---|---|---|---|
| Example | | PSD | PSD | PSD | (before | pH | | |
| # | TS | d(0.1) | d(0.5) | d(0.9) | UHT) | (FP) | Viscosity | Density |
| 8 | 22.15 | | | | 6.72 | 6.85 | 31.47 | 1.02 |
| 13 | 20.88 | 0.318 | 1.529 | 4.54 | 6.71 | 6.81 | 24.5 | |
| 16 | 20.63 | 0.542 | 1.962 | 5.063 | 6.78 | 6.83 | 30.4 | |
| 14 | 21.68 | 0.173 | 0.535 | 3.451 | 6.8 | 6.86 | 37.9 | |
| 15 | 21.33 | 0.167 | 0.485 | 3.154 | 6.7 | 6.86 | 32.5 | |
| 12 | 20.88 | 0.318 | 1.529 | 4.53 | 6.68 | 6.81 | 24.5 | |

### Example 19: Cellulose/Gel Gum (MCC 85%/15% CMC)+MPC at 7.5%, KOH was Added to Adjust pH

| | | | | pH | | | |
|---|---|---|---|---|---|---|---|
| | PSD | PSD | PSD | (before | pH | | |
| TS | d(0.1) | d(0.5) | d(0.9) | UHT) | (FP) | viscosity | density |
| 21.44 | 0.108 | 0.23 | 0.533 | 7.2 | 7.14 | 31.4 | 1.025 |

| Time | Temp | pH | Viscosity | Creaming | Serum | Sediment |
|---|---|---|---|---|---|---|
| 1 month | ambient | 7.03 | 35 | 0 | 4 | 1 |
| 1 month | fridge | 7.06 | 58.6 | 0 | 0 | 0 |
| 1 month | 35 C. | 6.98 | 40.6 | 0 | 4 | 1 |
| 1 month | 45 C. | 6.9 | 39.4 | 3 | 4 | 1 |
| 3 month | ambient | 6.94 | 22.9 | 2 | 3 | 2 |
| 3 month | fridge | 7.02 | 40.5 | 3 | 3 | 1 |
| 3 month | 35 C. | 6.84 | 30 | 2 | 4 | 2 |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A composition comprising:
medium chain triglycerides (MCTs) comprising from about 51 wt % to about 90 wt % of octanoic acid; and
at least one gum selected from the group consisting of xanthan gum, gellan gum, carrageenan (iota), carboxymethyl cellulose (CMC), and microcrystalline cellulose (MCC).

2. The composition of claim 1, wherein the at least one gum comprises gellan gum and iota carrageenan.

3. The composition of claim 1, wherein the at least one gum comprises at least one of carboxymethyl cellulose (CMC) or microcrystalline cellulose (MCC).

4-9. (canceled)

10. The composition of claim 1, wherein the composition further comprises at least 4 g protein/100 g of the composition.

11. The composition of claim 1, wherein the composition further comprises proteins in a weight ratio of at least 0.1 g protein/1.0 g of the MCTs.

12. The composition of claim 1, wherein the composition further comprises at least one ingredient selected from the group comprising (i) carbohydrates in a weight ratio of at least 0.1 g carbohydrate/1.0 g of the MCTs and (ii) lipids, other than the MCTs, in a weight ratio of at least 0.1 g lipids/1.0 g of the MCTs.

13. The composition of claim 1, wherein the composition has a pH from about 6.6 to about 7.5.

14. The composition of claim 1, wherein the composition is in a form selected from the group consisting of a beverage, mayonnaise, salad dressing, margarine, low-fat spread, dairy product, cheese spread, processed cheese, dairy dessert, flavoured milk, cream, fermented milk product, cheese, butter, condensed milk product, ice cream mix, soya product, pasteurised liquid egg, bakery product, confectionary product, confectionary bar, chocolate bar, high fat bar, liquid emulsion, spray-dried powder, freeze-dried powder, UHT pudding, pasteurised pudding, gel, jelly, yoghurt, a food with a fat-based or water-containing filling, and combinations thereof.

15-16. (canceled)

17. A method of improving sedimentation and/or mouthfeel attributes of a composition comprising medium chain triglycerides (MCTs), the method comprising:
providing to the composition at least one gum selected from the group consisting of xanthan gum, gellan gum, carrageenan (iota), carboxymethyl cellulose (CMC), and microcrystalline cellulose (MCC).

18. The method of claim 17, wherein the at least one gum comprises gellan gum and carrageenan iota.

19. The method of claim 17, wherein the at least one gum comprises at least one of carboxymethyl cellulose (CMC) or microcrystalline cellulose (MCC).

20-23. (canceled)

24. The method of claim 17, wherein the composition further comprises at least 1 wt % milk protein concentrate (MPC).

25-28. (canceled)

29. A method of improving cognitive functioning comprising at least one of episodic memory, executive function, or language skills of an individual in need thereof, the method comprising administering to the individual a composition comprising medium chain triglycerides (MCTs) and at least one gum selected from the group consisting of xanthan gum, gellan gum, carrageenan (iota), carboxymethyl cellulose (CMC), and microcrystalline cellulose (MCC).

30-32. (canceled)

33. The method of claim 29, wherein the at least one gum comprises gellan gum and iota carrageenan.

34. (canceled)

35. The method of claim 29, wherein the at least one gum comprises about 15 wt % carboxymethyl cellulose (CMC) and about 85 wt % microcrystalline cellulose (MCC).

36. The method of claim 29, wherein the composition is administered to the individual in a serving that provides at least about 5 g MCTs.

37. (canceled)

38. The method of claim 29, wherein the MCTs comprises from about 51 wt % to about 90 wt % of octanoic acid.

39-42. (canceled)

43. The method of claim 29, wherein the individual suffers from at least one of deficit in memory, impaired thinking skill comprising inability to make sound decisions and poor judgment, depression, or anxiety.

44. The method of claim 29, wherein the individual has or suffers from a brain energy deficiency condition or disease, neurological condition, and/or cognitive impairment.

45. The method of claim 29, wherein the individual has a condition selected from the group consisting of epilepsy, a neurological disease, a neurodegenerative disease, heart failure, inborn errors of metabolism, obesity, types 2 diabetes, nonalcoholic fatty liver disease (NAFLD), nonalcoholic steatohepatitis (NASH), cancer, a brain energy deficiency condition, a migraine, a memory disorder, an age-related memory disorder, a brain injury, a stroke, amyloid lateral sclerosis, multiple sclerosis, cognitive impairment, mild cognitive impairment (MCI), cognitive impairment post-intensive care, age-induced cognition impairment, Alzheimer's disease, Parkinson's disease, Huntingdon's disease, an inherited metabolic disorder, bipolar disorder, schizophrenia, and combinations thereof.
